(19) United States
(12) Patent Application Publication
(10) Pub. No.: US 2013/0188782 A1
(43) Pub. Date: Jul. 25, 2013

(54) OPTICAL ADJUSTMENT DEVICE

(71) Applicant: Thilo Hannemann, Erlangen (DE)

(72) Inventor: Thilo Hannemann, Erlangen (DE)

(21) Appl. No.: 13/747,908

(22) Filed: Jan. 23, 2013

(30) Foreign Application Priority Data
Jan. 23, 2012 (DE) 102012200893.4

(51) Int. Cl. A61B 6/00

(52) U.S. Cl. CPC A61B 6/583
USPC 378/207

(57) ABSTRACT

An optical adjustment device has a laser unit with at least one laser radiation source as well as an adjustment phantom which is arranged relative to the laser unit such that laser radiation emitted by the laser unit strikes the adjustment phantom. A fluorescent medium) is applied on the adjustment phantom, the fluorescent medium being designed to emit light of a different wavelength from the laser radiation upon being struck by laser radiation. This light is detected by a photodetector that is at a location spatially separated from the adjustment phantom, such as at the laser unit.

### BACKGROUND OF THE INVENTION

1. Field of the Invention

The present invention concerns an optical adjustment device, in particular at an imaging medical apparatus. The invention furthermore concerns a method to adjust a laser unit of an imaging medical apparatus, in particular of an apparatus to generate three-dimensional image data.

2. Description of the Prior Art

Optical adjustment devices at imaging medical apparatuses typically operate with laser radiation and are known from DE 195 01 069 A1 and DE 10 2008 013 615 A1, for example. In both cases, intersecting, fan-shaped beams are used for marking purposes. The radiation sources emitting the laser light—diode lasers, for example—can be attached to a component of the medical apparatus.

Computed tomography scanners and magnetic resonance tomography scanners (among others) are customarily used as imaging medical apparatuses. Such tomographic scanners serve for the generation of three-dimensional image data that are associated with a coordinate system. If a medical apparatus has an optical marking system, the spatial arrangement of components—in particular laser deflection units—of such a marking system can likewise be described with the use of a coordinate system. If it is necessary for a defined geometric relationship to be established between the coordinate system of the three-dimensional image data and the coordinate system of the optical marking system, a complicated adjustment of components is required.

### SUMMARY OF THE INVENTION

An object of the invention is to further develop an optical adjustment device relative to the prior art, in particular an optical adjustment device that is suitable for use in an imaging medical apparatus.

The invention proceeds from the insight that an adjustment phantom, which has known geometric properties and is irradiated (struck) by a laser beam from a laser unit that is arranged at the medical apparatus, can be used for the adjustment of an optical system at the imaging medical apparatus, which optical system also operates with laser radiation. The laser radiation striking the adjustment phantom could be, for example, detected by detectors that are placed on the surface of the phantom. The signals detected by the detectors directly indicate at which point of the adjustment phantom the (for example linear or fan-shaped) laser radiation strikes.

The invention deliberately rejects from such a direct detection of laser radiation by means of an adjustment phantom. Although an adjustment phantom is used according to the invention, it is not provided with detectors but rather with at least one marking made from a fluorescing medium. When it is exposed to the radiation of the laser unit, this marking emits light with a different wavelength in comparison to the radiation which is radiated by the laser unit. A photodetector that is spatially separate from the adjustment phantom is provided to detect the light emitted by the fluorescing marking on the adjustment phantom. The photodetector is preferably a photodiode, but in principle any detector that responds to the light emitted by the fluorescing medium is suitable. The adjustment phantom is designed and positioned such that it can also be recognized in the image generated by the imaging apparatus. The position of the phantom in the image coordinate system can thereby be correlated with the laser coordinate system and/or spatial coordinate system. The phantom therefore is composed of a suitable material (for example with high absorption for x-ray radiation) so that it is detectable as such in the image. The phantom is preferably a body (for example a sphere) made of suitable material that is provided with a coating that embodies the fluorescing medium.

According to a first embodiment, the adjustment phantom is arranged on the patient table of the imaging medical apparatus (in particular in the scanner (data acquisition unit) of a computed tomography). The apparatus adjustment phantom also includes structures (in particular point-shaped markings) that are detectable with the imaging medical apparatus, in particular by the x-ray radiation of the computed tomography apparatus. These structures are arranged in a known geometric relation to the fluorescing marking or the fluorescing markings. In particular, the fluorescing marking can be applied directly at the structures of the adjustment phantom that are detectable with the imaging medical apparatus.

According to a second embodiment, the adjustment phantom is an integral component of a patient table. For example, the fluorescent medium with geometrically defined (in particular point-shaped) structure is located on a facing side of a bed board, which forms an adjustable (in particular longitudinally displaceable and/or height-displaceable) component of the patient table.

In each of the cited embodiments, the fluorescent medium can be applied in the form of an at least approximately point-shaped marking (for instance in the shape of a circular disk, spherical cap or sphere) at the adjustment phantom. The diameter of such a marking is preferably 2 to 5 mm.

In a further embodiment, the adjustment phantom has a number of discrete (individually identifiable) point-shaped (in the cited sense) markings formed by fluorescent medium. For example, three point shaped markings (respectively formed by fluorescent medium) arranged at the vertices of an imaginary (in particular equilateral) triangle are applied to the adjustment phantom. These multiple (in particular three) markings represent a marking group. Upon scanning the marking group with a laser beam of known (for example fan-shaped) geometry, a characteristic signal (typically with multiple maxima) is acquired by the photodetector due to the fluorescing properties of the medium used for marking. Depending on the cross section geometry of the laser beam and the geometry of the marking group, the acquired signal structure can depend on the particular direction that the laser beam paints (spreads across) the marking structure. This correlation is usable by virtue of multiple marking groups of different geometry and/or alignment being applied on the adjustment phantom. In such cases the signal structure acquired with the photodetector unambiguously indicates which of multiple markings or marking groups on the adjustment phantom are struck by the laser beam. In the case of a fan-shaped laser beam, an identification of a marking group can optionally take place or be improved by repeatedly directing the laser beam that over the marking group in respectively different angular relationships to that marking group. A characteristic signal that is detectable by the photodetector results at each pass over the marking group.

A differentiation of different markings or marking groups on the adjustment phantom is possible not only through their geometry also (additionally or alternatively) through the use of fluorescent media with different optical properties. A fluorescent medium with a specific wavelength of the emitted light can be selected for each marking or marking group. Individual markings—in particular marking points—within a marking group can also have fluorescing materials with different optical properties, such that by detecting the wavelength of the emitted light it can be unambiguously established which of the markings is struck by the laser beam.

The function of the photodetector (fashioned as a photodiode, for example) that is provided to detect the light emitted by the fluorescent medium can be optimized by a color filter arranged in the beam path between the fluorescing marking and the photodetector (preferably placed immediately in front of the photodetector). The optical properties of the color filter are hereby selected such that they are matched to the properties of the fluorescing marking. If multiple markings with different optical properties are located on the adjustment phantom, one photodetector with a number of color filters can be used to detect the light emitted by these markings, for example. Multiple photodetectors each with a respective color filter can alternatively be used. In all cases, the photodetector—possibly with associated color filter—is designed so that it does not respond to the wavelength of the light radiated by the laser unit.

In an advantageous operating mode, the laser unit is operated in a modulated manner, and the signal detected by the photodetector is correlated with this modulation. The light signal radiated by the laser unit hereby has the shape of a square wave signal, for example. The clocking of the signal can take place with an arbitrary clock frequency and duration of the individual signals, for example such that a light signal of a specific, short duration always follows a signal pause of relatively longer duration. Light signals striking the at least one photodetector are further processed corresponding to the modulation of the laser unit only when the light signal can be a signal emitted by the fluorescing marking. In this way a possible influence of environmental brightness on the signal detection conducted with the photodetector is avoided (at least drastically reduced).

In principle, the photodetector can be located at an arbitrary location outside of the adjustment phantom. In particular, the photodetector can be attached to the imaging medical apparatus. The photodetector is advantageously combined into one structural unit with the laser unit or with a component of the laser unit, in particular a laser radiator and/or a laser deflection unit.

A particular advantage of the invention is that an adjustment phantom, which can be placed on a patient bed of a medical apparatus suitable for generation of three-dimensional image data (in particular computed tomography scanners or magnetic resonance tomographs), requires no detectors or other electrical or electronic components whatsoever. Rather, all components of the adjustment device that must be supplied with electrical energy and/or that deliver electrical signals are arranged outside of the adjustment phantom. In particular, a photodetector that detects light emanating from fluorescing markings on the adjustment phantom can be arranged at a laser unit attached to the computed tomography scanner, for example, can be connected with a laser light source.

### BRIEF DESCRIPTION OF THE DRAWINGS

FIG. 1 shows an imaging medical apparatus with an optical adjustment device having a laser unit and an adjustment phantom marked with a fluorescent marking in accordance with the invention.

FIG. 2 shows an alternative embodiment of an adjustment phantom in accordance with the invention.

FIG. 3 shows an embodiment of a fluorescent marking on an adjustment phantom in accordance with the invention.

FIG. 4 shows a signal curve of a signal generated by a photodetector of an adjustment device with a fluorescent marking according to FIG. 3.

### DESCRIPTION OF THE PREFERRED EMBODIMENTS

Components that correspond to one another are labeled with the same reference characters in all figures.

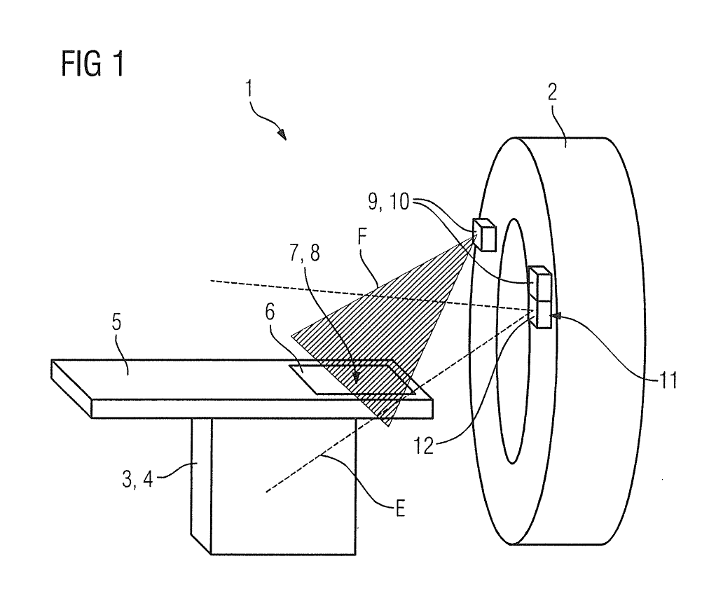

An imaging medical apparatus 1 that is only partially shown in FIG. 1 is a computed tomography scanner; the basic operation thereof being described in the cited prior art as well as DE 10 2010 015 060 A1 (for example).

A gantry 2 as well as a patient table 3 (which has a bed board 5 supported so as to be adjustable on a base 4) are apparent in FIG. 1 as components of the imaging medical apparatus 1. An adjustment phantom 6, which supports a point-shaped marking 7 made from a fluorescent medium 8, is arranged on the bed board 5. The adjustment phantom 6 serves for the adjustment of a laser unit 9 which is attached to the gantry 2 and comprises two laser radiation sources 10 as well as a photodetector 11. The photodetector 11 has a color filter 12 and acts in the following manner with the laser radiation sources 10 and the adjustment phantom 6:

As is shown in FIG. 1, a beam fan F which strikes the adjustment phantom 6, is radiated from at least one laser radiation source 10. The laser unit 9 is operated in a modulated manner so that laser light in the form of a beam fan F is radiated in short pulses. The attitude of the beam fan F is easily changed from pulse to pulse, such that the beam fan F is essentially continuously panned over the adjustment phantom 6.

The laser light striking the adjustment phantom 6 is at least partially reflected from the surface of the adjustment phantom 6 and strikes the photodetector 11 (fashioned as a photodiode), which is assembled together with one of the laser radiation sources 10 into a structural unit. Laser deflector units that are likewise integrated into the laser unit 9 are not separately perceivable in FIG. 1 and form components of the laser radiation sources 10.

The photodetector 11 (which is shown only at one of the laser radiation sources 10 in FIG. 1, wherein the second laser radiation source 10 can be equipped with a photodetector 11 in a corresponding manner) is designed to detect optical signals from a detection region E indicated with dashed lines. Due to the color filter 12 arranged immediately in front of the actual detector, the photodetector 11 detects no light of the frequency radiated by the laser unit 9. Rather, the photodetector 11 specifically detects the light emitted from the fluorescent medium 8 upon exposure with the laser light. Given beam fans F painting over the adjustment phantom 6, when the marking 7 is intercepted by the laser light is thus clearly detectable by means of the photodetector 11.

The adjustment phantom 6 thus satisfies its function without being equipped with active, electronic components. In particular, no wiring is required between the adjustment phantom 6 and the gantry 2.

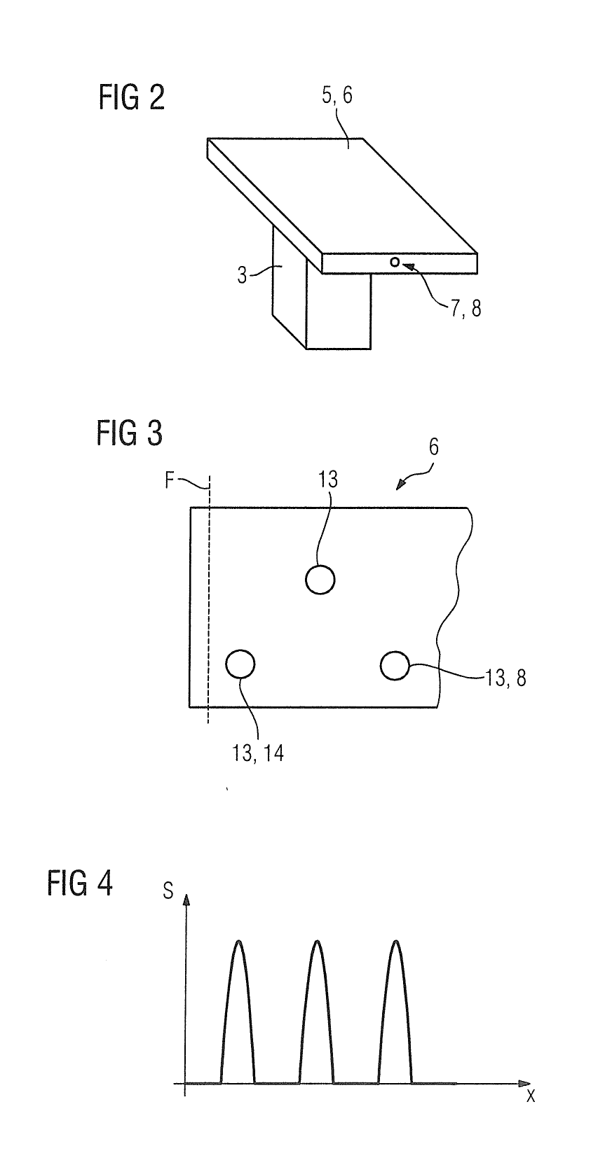

The exemplary embodiment according to FIG. 2 differs from the exemplary embodiment according to FIG. 1 in that the marking 7 formed from fluorescent medium 8 is applied directly to the patient table 3, namely on a facing side of the bed board 5. The adjustment phantom 6 is thus an integral component of the patient table 3. The direct application of the fluorescent marking 7 on the patient table 3 has the advantage that the marking 7 can remain at this point, and no intervention of an operator is required to implement the adjustment. The arrangement according to FIG. 2 is thus particularly suitable for automatic calibration. The diameter of the marking 7 (in the shape of a circular disc) is approximately 2 to 5 mm in the exemplary embodiment according to FIG. 2, just as in the exemplary embodiment according to FIG. 1.

A marking group 14 composed of three individual, similar markings 13 is shown in FIG. 3, which marking group 15 can be applied either directly on the bed board 5 (corresponding to the exemplary embodiment shown in FIG. 2) or on a separate adjustment phantom 6 resting on the patient table 3 (as shown in FIG. 1).

The three approximately point-shaped markings 13 (which respectively have a diameter of 2 to 5 mm) are arranged at the vertices of an imaginary, equilateral triangle. The optical properties of all three markings 13 can either be identical or differ from one another. In the latter cited case, by the measurement of the frequency of the light emitted from the marking 13 (which light arises by fluorescence) it can be unambiguously established which of the three markings 13 is directly intercepted by the beam fan F successively sweeping over the entire marking group 14 (also drawn in FIG. 3). In addition to the marking group 14 shown in FIG. 3, additional markings 7, 13 (either individual or assembled into at least one marking group 14) can be located on the adjustment phantom 6 (the manner is not shown). Such additional markings 7, 13 can also differ from the markings 13 visible in FIG. 3 (in the form of a triangle) not only in geometric features but also in their optical properties.

The diagram shown in FIG. 4 shows the signal strength S acquired by the photodetector 11 (which signal strength S is dependent on the location of the radiation of the laser light onto the adjustment phantom 6, i.e. on the panning of the beam fan F), wherein the marking group 14 shown in FIG. 3 as well as the relationship shown there between the alignment of the beam fan F and the marking group 14 is considered. Due to the arrangement of the markings 13 forming the marking group 14 at the vertices of an equilateral triangle, and the alignment of the line illuminated by the beam fan F orthogonal to the base of this triangle, the peaks visible in the diagram according to FIG. 4 are spaced equidistantly from one another. If the beam fan F were rotated relative to the marking group 14 in comparison to the configuration according to FIG. 3, the distances between the peaks in the diagram according to FIG. 4 would change. The shape of the fluorescent marking 7 that is shown in FIGS. 1 and 2 generates an entirely different signal pattern, namely a single maximum. These effects can be used in order to automatically differentiate different markings 7, 13 or marking groups 14 which are located on the adjustment phantom 6 from one another by means of the photodetector 11.

Although modifications and changes may be suggested by those skilled in the art, it is the intention of the inventor to embody within the patent warranted hereon all changes and modifications as reasonably and properly come within the scope of his contribution to the art.

I claim as my invention:

1. An optical adjustment device, comprising:
a laser unit comprising at least one laser radiation source that emits laser radiation;
an adjustment phantom located at a position relative to said laser unit that causes said adjustment phantom to be struck by said laser radiation; and
said adjustment phantom comprising a phantom body with a fluorescent medium applied thereon, said fluorescent medium being composed of a material that emits light, when struck by said laser radiation, of a different wavelength than a wavelength of said laser radiation; and
a photodetector located remote from said adjustment phantom that detects light emitted by said fluorescent medium.

2. An adjustment device as claimed in claim 1 wherein said adjustment phantom is configured to be arranged on a patient table of a medical imaging apparatus.

3. An adjustment device as claimed in claim 1 wherein said adjustment phantom is integrated into a patient table of a medical imaging apparatus.

4. An adjustment device as claimed in claim 1 wherein said fluorescent medium is applied on said phantom body as at least one point-shaped marking.

5. An adjustment device as claimed in claim 1 wherein said fluorescent medium is applied on said phantom body as a plurality of point-shaped markings.

6. An adjustment device as claimed in claim 5 wherein said fluorescent medium comprises three of said point-shaped markings respectively located at vertices of a triangle on said phantom body.

7. An adjustment device as claimed in claim 5 wherein at least two of the point-shaped markings in said plurality of point-shaped markings had respectively different optical properties.

8. An adjustment device as claimed in claim 1 wherein said photodetector is a photodiode.

9. An adjustment device as claimed in claim 1 wherein said photodetector is combined with said laser unit as a single structural unit.

10. An adjustment device as claimed in claim 1 comprising a color filter located in front of said photodetector.

11. An adjustment device as claimed in claim 1 wherein said fluorescent medium comprises a plurality of point-shaped markings on said phantom body, with at least two of said point-shaped markings in said plurality of point-shaped markings having respectively different optical properties, and wherein said photodetector comprises a plurality of individual photodetectors, with at least two of said photodetectors respectively having different optical filters in front thereof, said different optical filters being respectively matched to the different optical properties of said at least two of said point-shaped markings.

12. A method to adjust a laser unit of a medical imaging apparatus, comprising:
providing an adjustment phantom having at least one marking thereon formed by a fluorescent medium;
irradiating said fluorescent medium on said adjustment phantom with laser radiation having a known beam geometry and having a laser radiation wavelength, to cause said fluorescent medium to emit light by fluorescence having a wavelength that is different from said laser radiation wavelength; and
detecting said light emitted by said fluorescent medium with a photodetector.

13. A method as claimed in claim 12 comprising irradiating said adjustment phantom with laser radiation having a fan-shape.

14. A method as claimed in claim 12 comprising modulating emission of said laser radiation, and correlating the light detected by said photodetector with the modulation of said laser radiation.
